(19) United States
(12) Patent Application Publication
(10) Pub. No.: US 2024/0016399 A1
(43) Pub. Date: Jan. 18, 2024

(54) METHOD AND MEASURING DEVICE FOR CONTINUOUSLY NON-INVASIVELY DETERMINING AT LEAST ONE CARDIOVASCULAR PARAMETER

(71) Applicant: CNSYSTEMS MEDIZINTECHNIK AG, GRAZ (AT)

(72) Inventor: Jürgen Fortin, Graz (AT)

(21) Appl. No.: 18/036,741

(22) PCT Filed: Nov. 9, 2021
(86) PCT No.: PCT/AT2021/060422

§ 371 Date: May 12, 2023

(30) Foreign Application Priority Data
Nov. 12, 2020 (AT) A 50986/2020

(51) Int. Cl. A61B 5/022; A61B 5/00

(52) U.S. Cl. CPC A61B 5/02241; A61B 5/6826; A61B 5/02422

(57) ABSTRACT

The invention relates to a method and a measuring device for continuously non-invasively determining at least one cardiovascular parameter, preferably the arterial blood pressure, at an extremity containing an artery, the measuring device comprising a receiving element that can be attached to the extremity and is suited to at least partly surround the extremity, and comprising a flexible bubble which is supported on the receiving element, acts on the extremity and is filled with a fluid. According to the invention, an actuator which is suited to vary the pressure in the flexible bubble is placed in or on the receiving element, and the flexible bubble includes a pressure sensor which is in contact with the fluid in the flexible bubble and which is suited to continuously measure the absolute pressure value. The measuring device further comprises a unit suited to measure the pulsations generated by the volume flow in the artery, and a control unit having two different modes of operation, i.e. a measuring phase and an interpolation phase.

### CROSS REFERENCE TO RELATED APPLICATIONS

The present application is a U.S. National Phase Application pursuant to 35 U.S.C. § 371 of International Application No. PCT/AT2021/060422 filed Nov. 9, 2021, which claims priority to Austrian Patent Application No. A 50986/2020 filed Nov. 12, 2020. The entire disclosure contents of these applications are herewith incorporated by reference into the present application.

### BACKGROUND

The invention relates to a method and a measuring device for continuously, non-invasively determining at least one cardiovascular parameter, preferably arterial blood pressure, on an extremity containing an artery, comprising a recording element, which can be attached to the extremity and is suitable for at least partially enclosing the extremity, and comprising a flexible, fluid-filled bladder, which is supported on the recording element and acts on the extremity.

The continuous, non-invasive measurement of cardiovascular parameters, in particular blood pressure, has to date been a major challenge for measurement technology. For decades, one focus of research has been on so-called “cuffless” or “ubiquitous” measuring methods and devices. These methods measure various pulsatile body signals without exerting a bothersome pressure on the body by way of a cuff. Particularly since the breakthrough of various “smart” sensors, such as fitness straps and smart watches, but also smart textiles or body scales, etc., there has been a desire to acquire other also cardiovascular parameters besides the pulse rate.

These methods usually calculate blood pressure from time differences that can be derived from signals derived from at least two different parts of the body. Specifically, the time taken by a pulse to travel from a distal part of the body to a proximal part of the body is measured. In the literature, this time is referred to as the “pulse transit time” or “pulse arrival time”.

U.S. Pat. No. 8,100,835 B2 describes a so-called “pulse decomposition analysis”, which breaks down the pulse into a forward and a backward pulse wave. The time differences are measured and are said to be a measure of the blood pressure. The advantage of this method is that it requires the use of just one sensor.

There are also methods that aim to calculate the blood pressure from the pulsatile signals of a single sensor. US 2017 0360314 A1 describes a method and a device in which the blood pressure can be continuously determined from the measurement of the pulse wave. Also increasingly being published in the scientific literature are methods which aim to determine the blood pressure from a single sensor by means of machine learning or other Artificial Intelligence methods.

All these “cuffless” measuring methods have at least two disadvantages. These methods cannot determine the absolute value of the blood pressure and thus usually have to be calibrated to the blood pressure measured using a cuff on the upper arm or wrist. Furthermore, other physiological events change both the time differences and the shapes of the pulse waves, without any change in blood pressure occurring. This results in changes to the mathematical model on which the calculation of blood pressure is based, and the results are falsified. This is caused by changes to the vascular resistance by the smooth vascular muscles, which can itself open (vasodilation) as well as close (vasoconstriction). This physiological phenomenon is continuously controlled by the vegetative nervous system and implies that the calibration interval has to be quite short for these measuring methods.

WO 2020 176206 A1 describes a system in which a calibration can be carried out using an arm cuff. However, this method and the associated devices require two sensors, namely the pulsation sensor and the arm cuff.

US 2019 0059825 A1 describes a self-calibrating system using a pneumatic finger cuff. Essentially, a so-called “oscillometric” measurement is performed intermittently on the finger by means of the air-filled bladder (cuff), and these values can then be used to calibrate a system, preferably a system that operates using “pulse decomposition analysis”.

On account of the above-mentioned disadvantages of the “cuffless” methods, the so-called “vascular unloading technique” is beginning to prevail on the market; this technique can be traced back to a publication by Periaz (Digest of the 10th International Conference on Medical and Biological Engineering 1973 Dresden), in which light is shone through a finger and the recorded flow is kept constant by a servo control.

Patent EP 2 854 626 B1 describes a novel method for the so-called “vascular control technique”, including the associated device, which applies an only very slowly changing contact pressure to the extremity (usually a finger) in order thus to monitor the mean arterial blood pressure. U.S. Pat. No. 10,285,599 A1 describes various measurement modes and supplementary elements that are important for use as a wearable device.

Both in the “vascular unloading technique” and in the “vascular control technique”, pressure is continuously exerted, usually on a finger, during the measurement. EP 1 179 991 B1 describes, inter alia, a double finger sensor in which two adjacent fingers can alternately be acted upon by pressure and measured. EP 3 419 515 B1 likewise describes a double finger system, in which the two adjacent fingers come to lie on a body that resembles a computer mouse. In this way, the measurement can be carried out on one finger, while the other finger rests.

U.S. Pat. No. 10,285,599, which is mentioned above, describes a measurement mode for the “vascular control technique” in which the pressure on an extremity (e.g. a finger) is reduced to around 30-mmHg following a measurement, and only the heart rate continues to be measured. The finger can therefore rest while waiting for the next measurement. However, this has the disadvantage that no complete cardiovascular values are available during this so-called “idle phase”.

The medRxiv preprint bearing the title “A novel art of continuous non-invasive blood pressure measurement” (FORTIN et al.) discloses a sensor, wearable on a finger, for continuously measuring the blood pressure (BP) and derived cardiovascular variables. This is a compact measuring device for continuously, non-invasively monitoring arterial blood pressure. The pulsating blood pressure signal that is measured contains information for deriving cardiac output and other hemodynamic variables.

### SUMMARY

The object of the invention is to build a measuring device and a method for continuously, non-invasively determining at least one cardiovascular parameter, preferably arterial blood pressure, on an extremity in such a way as to enable continuous determination of the parameters, the aim being to avoid long-lasting compressive loads for the extremity to be measured. The aim is also to provide a compact system consisting of few individual parts, which can even be integrated in a wearable unit.

This object is achieved by a measuring device according to claim 1 and a measuring method according to claim 6. Advantageous embodiment variants are disclosed in the dependent claims.

The present application describes a measuring method and a measuring device by which all cardiovascular values of a person can be determined continuously, even though pressure is exerted on an extremity (e.g., a finger) only relatively briefly during a measuring process.

The method according to the invention has in principle two different operating modes. First, a measurement phase is carried out, during which the pressure on the extremity in the sensor of the blood pressure measuring device can vary. An absolute value or the absolute values of the blood pressure are measured, and subsequently all necessary cardiovascular parameters are determined.

These cardiovascular parameters are at least the arterial blood pressure as a continuous pulsatile signal p_{A}(t), as well as the systolic (sBP), diastolic (dBP) and mean arterial blood pressure (mBP) for each heartbeat. Other cardiovascular values (such as e.g. cardiac output (CO), stroke volume (SV), systemic vascular resistance (SVR), etc.), dynamic variables (such as e.g. pulse pressure variation PPV or stroke volume variation SVV) or parameters of the vegetative/autonomic nervous system (such as e.g. baroreceptor reflex sensitivity BRS, blood pressure or heart rate variability BPV/HRV, etc.) can optionally also be determined.

During the measurement phase, a mathematical model is fed with the measured cardiovascular values and is calibrated using these values. The mathematical model may exist in various forms. On the one hand, the model may be built from experimentally determined a-priori knowledge, and the values obtained from the measurement phase parameterize the existing model. On the other hand, the model may be built from the measured values themselves using machine learning methods. Of course, all hybrid forms are also possible.

After the measurement phase, the second part of the method begins: Once the mathematical model has been determined with sufficient accuracy, the contact pressure in the blood pressure measuring device is reduced to a minimum value that is sufficient to continue to record the pulsations which occur as a result of the volume flow in the artery. By lowering the pressure, the amplitude of the pulsations changes, but so does the shape of the pulsations. The pulsations are fed to the mathematical model, and the model estimates or interpolates new cardiovascular parameters therefrom. These parameters can thus be determined without a bothersome pressure having to be exerted on the extremity.

The mathematical model is also able to determine possible errors in relation to the real cardiovascular parameters determined in a measurement phase. If the error becomes too large, then a new measurement phase is started in the blood pressure measuring device, during which, once again, a pressure is exerted on the extremity in the sensor of the blood pressure measuring device. A new measurement phase may also be started after a certain period of time. In a new measurement phase, the mathematical model may be completely rebuilt. However, parts of the model from the past measurement phase may also be reused, for example to shorten the time for machine learning and thus the measurement phase.

Another advantage of the present invention is that it requires the use of just one single sensor, ideally a wearable sensor.

### BRIEF DESCRIPTION OF THE FIGURES

The invention will be explained in greater detail below on the basis of schematic illustrations and diagrams:

FIG. 1—shows a measuring device according to the invention for determining arterial blood pressure, in a schematic illustration;

FIG. 2—shows a block diagram of a method according to the invention for determining arterial blood pressure, alternating between a measurement phase and an interpolation phase;

FIG. 3—shows a variant of the measuring device according to FIG. 1, in which the pulsatile component of the pressure is determined by means of photoplethysmography;

FIG. 4—shows a variant of the measuring device according to FIG. 1, in which the absolute value of the blood pressure is obtained from an oscillometric signal; and

FIG. 5—shows a block diagram of a variant of the method according to the invention with an initial phase.

### DETAILED DESCRIPTION

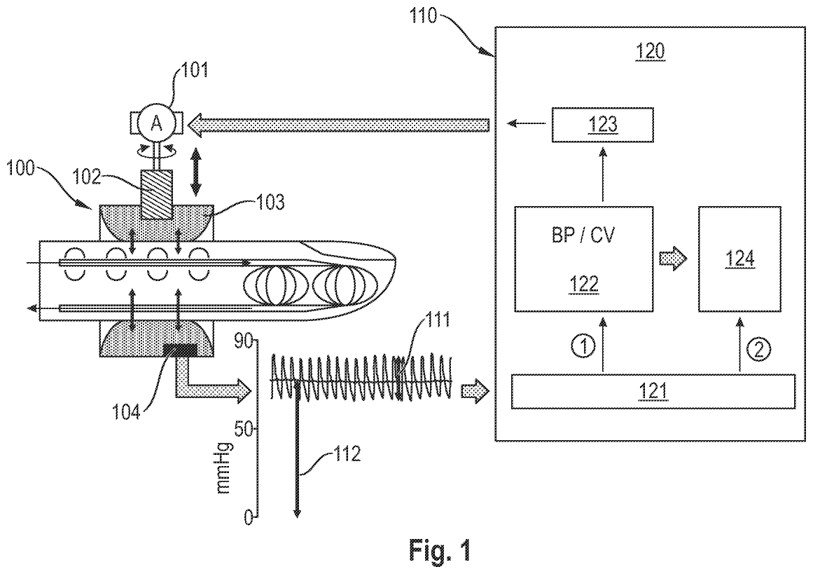

FIG. 1 shows an embodiment of the measuring device according to the invention for continuously, non-invasively determining at least one cardiovascular parameter, for example a blood pressure measuring device, which in this case is attached to the finger of one hand by means of a recording element 100 (e.g. a finger cuff). The measuring device substantially consists of an actuator 101 which, by way of a plunger or piston 102, exerts a pressure on a flexible bladder 103, which is arranged in the recording element 100. The bladder 103 is preferably filled with a liquid or a gas so that the pressure in the bladder 103 can also act on the finger and then on the artery in the finger.

The pressure in the bladder 103 is measured by means of a pressure sensor 104. In the present embodiment, this is a high-resolution pressure sensor 104, which can also act as a pressure sensor for the arterial pulses or the pulsatile component of the pressure signal. To this end, the pressure sensor 104 must have a sufficient resolution and must be able to sense changes in pressure of at least 0.01 mmHg (0.013 mbar) with an upper cut-off frequency of at least 40 Hz.

This present method works very well when the flexible bladder 103 is preferably filled with an incompressible fluid, for example a liquid. However, the pulsations can also be sufficiently transmitted by gas (e.g., air). In embodiment variants using an air-filled bladder 103, an air pump and one or more valves (not shown) may be required instead of a single plunger 102.

The pressure sensor 104 thus measures the absolute value 112 of the pressure in the bladder 103 and also the arterial pulsations or the pulsatile component 111 of the pressure signal. In an electrical equivalent, the absolute value 112 of the pressure corresponds to the direct component (DC) and the arterial pulsations 111 correspond to the alternating component (AC) of the pressure sensor signal. The signal is then fed to the control unit 110 of the blood pressure measuring device; in the present embodiment, this is a microcontroller 120.

The microcontroller 120 includes at least the following elements: computer unit or microcomputer, memory for the program code, working memory, analog-to-digital converter, digital-to-analog converter, components for voltage generation, and others. By way of example, use may be made of a microcontroller which already provides most of the functions integrated in one component. However, the controller may also be constructed using other methods, for example such as analog circuits.

In the microcontroller 120, the following elements are preferably mapped in a software code: signal detector 121, measuring unit 122 for the blood pressure BP and the other cardiovascular parameters CV, a control unit 123 for the actuator 101, and a mathematical model 124. In addition, input and output elements (not shown here) may be provided for operating the device.

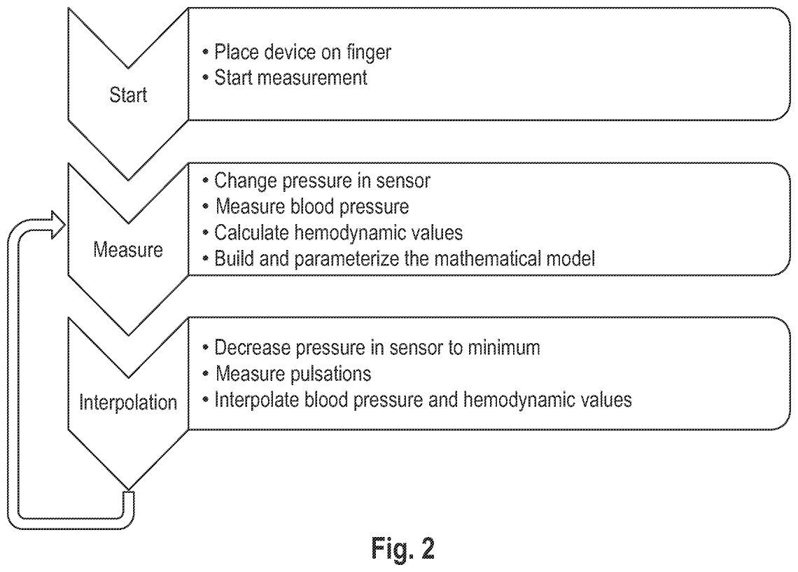

FIG. 2 shows a simple flow logic of the measuring method according to the invention: In a start phase, the blood pressure measuring device is attached to the extremity. Thereafter, the measurement can be started. In the measurement phase @, the pressure in the flexible bladder 103 is varied, with both the absolute value 112 of the pressure in the bladder 103 and the arterial pulsations 111 being measured by the pressure sensor 104.

To determine the blood pressure in the artery of the extremity, use can be made of known methods such as the “vascular control technique”, the “vascular unloading technique” or even the simple oscillometric method. The other cardiovascular parameters mentioned above can then also be determined from the blood pressure using known methods. The control of the blood pressure measuring method is preferably mapped in the measuring unit 122 for the blood pressure and the other cardiovascular parameters of the microcontroller 120 in the form of a software code. The pressure in the flexible bladder 103 and in turn on the finger is varied via a control unit 123 for the actuator 101.

These measured cardiovascular parameters are fed to a mathematical model 124. The mathematical model 124 may exist in various forms. On the one hand, the model 124 may be built from experimentally determined a-priori values, and the values obtained from the measurement phase \textcircled{1} parameterize the existing model. On the other hand, the model 124 may be built from the measured values themselves using machine learning methods. Of course, all hybrid forms are also possible. In addition to the measured cardiovascular parameters, the pressure signal from the pressure sensor 104, in particular the absolute pressure 112 and the pulsatile component 111 of the pressure signal, may also be fed to the mathematical model 124.

After the measurement phase \textcircled{1}, the second part of the method begins: the interpolation phase \textcircled{2}. Once the mathematical model 124 has been determined with sufficient accuracy, then the contact pressure and thus the absolute pressure 112 in the blood pressure measuring device is reduced to a minimum value. Preferably, the level of the contact pressure in the interpolation phase \textcircled{2} should be high enough that the pulsations 111 that occur as a result of the volume flow in the artery can continue to occur and be determined. Ideally, the contact pressure in the interpolation phase \textcircled{2} moves toward zero or is zero, so that the sensor does not bother the patient.

By lowering the absolute pressure 112, the amplitude of the pulsations or of the pulsatile component 111 of the signal changes, but so does the shape of the pulsations 111; however, certain properties such as time intervals, frequency contents, segments and sections of the pulse, etc. remain at least similar. The pulsations 111 are fed to the mathematical model 124, and the model “estimates” or interpolates new cardiovascular parameters therefrom. Here, “estimates” indicates that machine learning methods or methods from the “Artificial Intelligence” field may be used. These parameters can thus be determined without a long-lasting, bothersome pressure having to be exerted on the extremity.

The mathematical model is also able to determine possible errors in relation to the real cardiovascular parameters determined in a measurement phase \textcircled{1}. If the error becomes too large, then a new measurement phase \textcircled{1} is started in the blood pressure measuring device, during which, once again, a pressure is exerted on the extremity in the sensor of the blood pressure measuring device. A new measurement phase \textcircled{1} may also be started after a certain period of time. In a new measurement phase \textcircled{1}, the mathematical model may be completely rebuilt. However, parts of the model from the past measurement phase \textcircled{1} may also be reused in order thus, for example, to shorten the time for machine learning and thus the measurement phase \textcircled{1}.

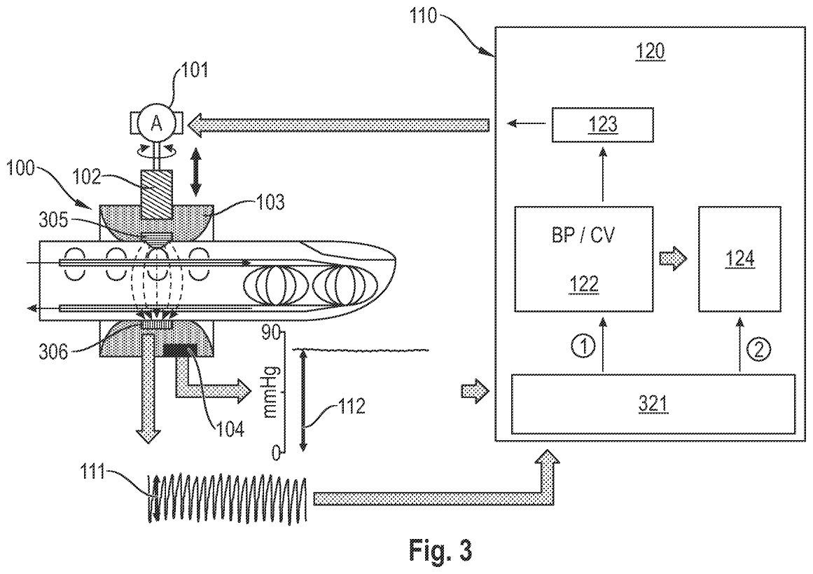

FIG. 3 shows a further embodiment of the measuring device according to the invention. It differs substantially in the measurement of the pulsations, or of the pulsatile component 111, which in this variant are measured by means of light sensors. These light sensors are mounted at the point where the flexible bladder 103 bears against the finger, and they consist of at least one light source 305 and at least one light detector 306. The light source 305 is preferably an LED with infrared light and emits through the finger. The infrared light is absorbed by the erythrocytes in the artery and, depending on the amount of erythrocytes, a modulated light is produced which emerges on the other side of the finger. The light detector 306 is preferably a photodiode and measures the modulated light radiating through the finger. This light is thus a measure of the volume of blood in the artery. This light signal, which represents the pulsations 311, is fed to the signal detector 121 of the microcontroller 120.

As in the embodiment variant according to FIG. 1, however, a pressure sensor 104 must also be present here, but this only needs to measure the absolute value 112 of the pressure in the flexible bladder 103 and feed it to the microcontroller 120.

The use of light sensors 305 and 306 has the advantage that the contact pressure during the interpolation phase \textcircled{2} can be reduced even further toward zero since, in theory, the pulsations caused by the changes in volume of the artery, which is not influenced by the contact pressure, may occur through the light. On the other hand, without contact pressure, it is difficult for the light sensors 305 and 306 to couple the light in and out through the skin. A contact pressure is present even in the so-called “cuffless” or “ubiquitous” measuring methods mentioned above, which mostly operate using light sensors. These sensors are often attached to the body by means of a strap (e.g. fitness watch), a spring or a hook-and-loop fastener in order to ensure that the light is coupled in and out.

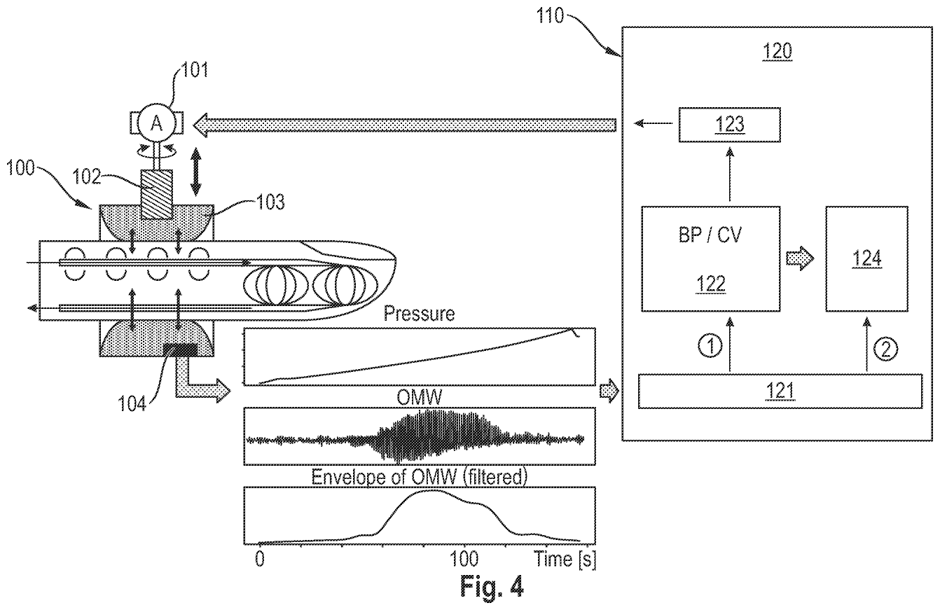

The embodiment variant of FIG. 1 is shown once again in FIG. 4. Here, however, the blood pressure is not measured continuously. The measuring unit 122 for the blood pressure BP and other cardiovascular parameters CV applies, for example, a pressure ramp to the finger (see sub-figure “Pressure”), and an oscillometric signal “OMW” or the “Envelope of the OMW” (see middle and bottom sub-figure, respectively) is determined. The systolic, diastolic and mean arterial blood pressure can be determined therefrom in a known manner. In principle, the mathematical model 124 could already be fed by this method in order to determine at least the blood pressure in the subsequent interpolation phase \textcircled{2}. This simple variant also belongs to the subject matter of the present application.

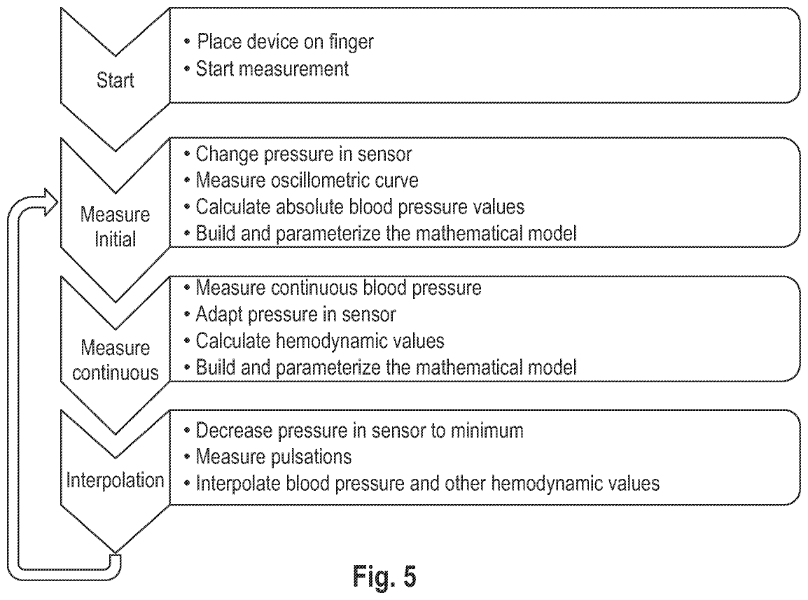

This oscillometric method can also be carried out initially at the beginning of a measurement phase \textcircled{1}, as shown in the flow diagram in FIG. 5. The values from this initial measurement phase can be used to control and calibrate a second, continuous measurement phase in order to be able to measure the cardiovascular values even more accurately. Here, the mathematical model 124 is created and parameterized both in the initial measurement phase and in the second, continuous measurement phase so that, in the interpolation phase \textcircled{2} the cardiovascular values can be determined using this model 124, without any pressure being exerted on the extremity.

1-15. (canceled)

16. A measuring device for continuously, non-invasively determining at least one cardiovascular parameter, preferably arterial blood pressure, on an extremity containing an artery, the measuring device comprising:
a recording element configured to be attached to the extremity and to at least partially enclose the extremity;
a flexible, fluid-filled bladder supported on the recording element and acting on the extremity;
an actuator positioned in or on the recording element, wherein the actuator is configured to vary the pressure in the flexible, fluid-filled bladder,
wherein the flexible, fluid filled bladder has a pressure sensor in contact with the fluid in the flexible, fluid filled bladder, the pressure sensor configured to continuously measure an absolute value of the pressure;
wherein the measuring device includes a component configured to measure pulsations that occur as a result of volume flow in the artery; and
wherein the measuring device has a first control unit including at least the following elements:
a signal detection unit configured to record the absolute value of the pressure and the pulsations that occur as a result of the volume flow in the artery;
a measuring unit configured to determine the at least one cardiovascular parameter;
a second control unit for the actuator configured to vary the pressure in the flexible, fluid filled bladder; and
a mathematical model configured to interpolate the at least one cardiovascular parameter based on the pulsations that occur as a result of the volume flow in the artery, while a pressure in the flexible, fluid filled bladder is reduced to a minimum during an interpolation phase of the measuring device.

17. The measuring device according to claim 16, wherein the first control unit has at least two different operating modes: a measurement phase and the interpolation phase.

18. The measuring device according to claim 16, wherein the pressure sensor is further configured to measure the pulsations that occur as a result of the volume flow in the artery.

19. The measuring device according to claim 16, wherein the component configured to measure the pulsations that occur as a result of the volume flow in the artery comprises a photoplethysmographic system having at least one light source and at least one light detector.

20. The measuring device according to claim 16, wherein the recording element, the flexible, fluid filled bladder, and the actuator are integrated in a unit configured to be worn on the body.

21. The measuring device according to claim 20, wherein the unit is configured to be worn on a finger of a hand.

22. A method for continuously, non-invasively determining at least one cardiovascular parameter on an extremity containing an artery, wherein the extremity is at least partially enclosed by a flexible, fluid-filled bladder, and wherein a pressure sensor, which generates a pressure signal p_{c}(t), is arranged in the flexible, fluid-filled bladder, the method comprising:
varying, by an actuator, a pressure in the flexible, fluid-filled bladder;
varying and measuring an absolute value of the pressure in the flexible, fluid-filled bladder in a measurement phase;
measuring pulsations generated by a volume flow in the artery during the measurement phase;
determining the at least one cardiovascular parameter from the absolute value and the pulsations;
feeding the at least one cardiovascular parameter to a mathematical model; and
subsequently, in an interpolation phase:
reducing the pressure in the flexible, fluid-filled bladder to a minimum,
measuring the pulsations generated by the volume flow in the artery during the interpolation phase;
feeding the pulsations that occur as a result of the volume flow in the artery to the mathematical model; and
interpolating the at least one cardiovascular parameter from the mathematical model and the pulsations that occur as a result of the volume flow in the artery.

23. The method according to claim 22, wherein the mathematical model is configured to calculate deviations and errors of the interpolation of the at least one cardiovascular parameter.

24. The method according to claim 23, wherein a restart of the measurement phase is initiated as a function of the calculated error of the at least one cardiovascular parameter.

25. The method according to claim 22, wherein a restart of the measurement phase is initiated after a specifiable period of time has elapsed.

26. The method according to claim 22, wherein the pulsations that occur as a result of the volume flow in the artery are fed to the mathematical model during the measurement phase.

27. The method according to claim 22, wherein the absolute value of the pressure is fed to the mathematical model during the measurement phase.

28. The method according to claim 22, wherein the vascular control technique (VCT) is used to determine the at least one cardiovascular parameter.

29. The method according to claim 22, wherein the vascular unloading technique is used to determine the at least one cardiovascular parameter.

30. The method according to claim 22, wherein an oscillometric method is used to determine the at least one cardiovascular parameter.

31. The method according to claim 22, wherein first the oscillometric method and then the vascular control technique or the vascular unloading technique is carried out to determine the at least one cardiovascular parameter.
